(19) United States
(12) Patent Application Publication
(10) Pub. No.: US 2005/0209542 A1
(43) Pub. Date: Sep. 22, 2005

(54) Tissue approximation sling and method

(76) Inventors: Daniel Irwin Jacobs, Palo Alto, CA (US); Greg Lamps, Sunnyvale, CA (US); Robert Elson, Los Altos Hills, CA (US)

Correspondence Address: DLA PIPER RUDNICK GRAY CARY US, LLP, 2000 UNIVERSITY AVENUE, E. PALO ALTO, CA, 94303-2248, US

(21) Appl. No.: 11/076,661

(22) Filed: Mar. 9, 2005

Related U.S. Application Data
(60) Provisional application No. 60/553,782, filed on Mar. 16, 2004.

(51) Int. Cl. A61F 5/00

(52) U.S. Cl. 602/16

(57) ABSTRACT

A medical sling device having a pair of tissue approximating devices each including a supportive backing member, a plurality of tissue engagement tines extending from the backing member, and an elongated extension member extending from the backing member. Each extension member is slidably engaged with the back member of the other tissue approximating device, such that pulling on the first and second extension members causes the first and second backing members to slide toward each other.

This application claims the benefit of U.S. Provisional Application No. 60/553,782, filed Mar. 16, 2004.

### FIELD OF THE INVENTION

The present invention relates to surgical devices and procedures, and more particularly to a tissue approximation device and method for supporting the repair of tissue such as the platysma muscle in rejuvenation surgery of the face.

### BACKGROUND OF THE INVENTION

The platysma is a facial muscle in the neck region. It is a fanlike muscle that originates from the fascia of the cervical region and inserts in the mandible and the skin around the mouth. It is innervated by the cervical branch of the facial nerve, and acts to wrinkle the skin of the neck and to depress the jaw.

Rejuvenation surgery of the neck includes a need for approximating the decussated edges and supporting the platysma muscle. However, using sutures alone for such approximation and support has a propensity to fail.

### SUMMARY OF THE INVENTION

The present invention provides a medical device and procedure for approximating the anterior edges and supporting tissue such as the platysma muscle using an adjustable multipoint tension distribution device.

More specifically, the present invention is a medical sling device that includes a first tissue approximation device that includes a first supportive backing member, a first plurality of tissue engagement tines extending from the first backing member, and a first elongated extension member extending from the first backing member, and a second tissue approximation device that includes a second supportive backing member, a second plurality of tissue engagement tines extending from the second backing member, and a second elongated extension member extending from the second backing member, wherein the first extension member is slidably engaged with the second backing member, and the second extension member is slidably engaged with the first backing member, such that tension pulling on the first and second extension members causes the first and second backing members to slide toward each other.

In another aspect of the present invention, a method of approximating first and second tissue portions uses a medical sling device, wherein the sling device comprises a first tissue approximation device that includes a first supportive backing member, a first plurality of tissue engagement tines extending from the first backing member, and a first elongated extension member extending from the first backing member; and a second tissue approximation device that includes a second supportive backing member, a second plurality of tissue engagement tines extending from the second backing member, and a second elongated extension member extending from the second backing member; wherein the first extension member is slidably engaged with the second backing member, and the second extension member is slidably engaged with the first backing member. The method comprises placing the sling device on the first and second tissue portions such that the first plurality of tines engage with a first tissue portion and the second plurality of tines engage with the second tissue portion, pulling on the first and second extension members such that the first and second backing members slide toward each other drawing the first and second tissue portions toward each other, and securing the first and second extension members in place.

Other objects and features of the present invention will become apparent by a review of the specification, claims and appended figures.

### BRIEF DESCRIPTION OF THE DRAWINGS

FIG. 1A is a top view of the tissue approximation sling of the present invention.

FIG. 1B is a side view of the tissue approximation sling of the present invention.

FIG. 2A is a perspective view of the tissue approximation devices of the present invention.

FIG. 2B is a perspective view of the supportive backing and tissue engagement tines of the tissue approximation devices of the present invention.

FIG. 3 is a front view showing the implementation of the tissue approximation sling of the present invention on the platysma muscle area of the neck.

FIG. 4 is a side view of an alternate embodiment of the tissue approximation sling of the present invention.

FIGS. 5A and 5B are perspective views of the alternate embodiment for the supportive backing and tissue engagement tines of the present invention.

FIGS. 6A and 6B are perspective views of the alternate embodiments for the tissue approximation sling of the present invention.

### DETAILED DESCRIPTION OF THE PREFERRED EMBODIMENTS

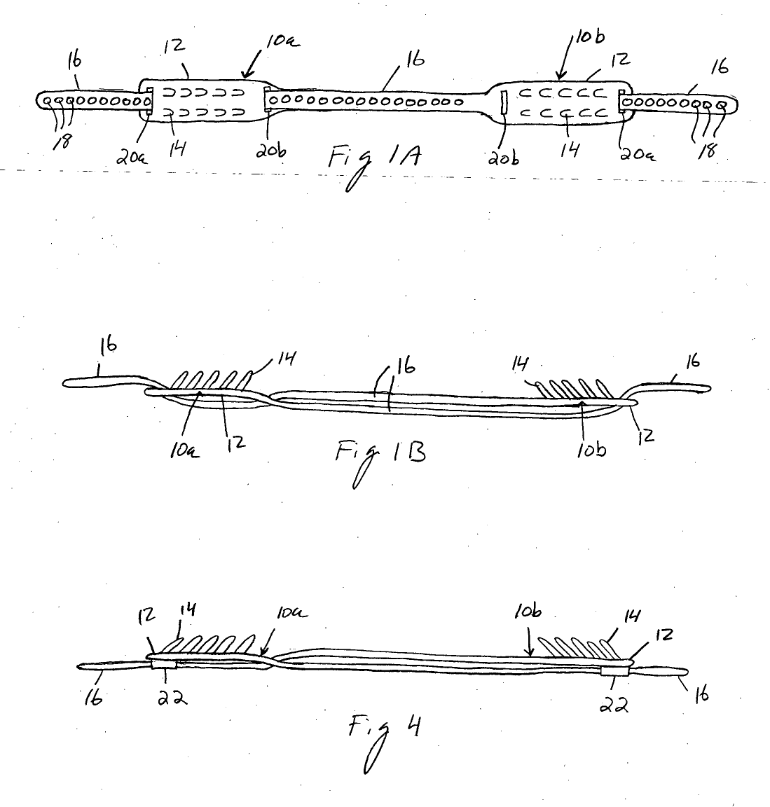

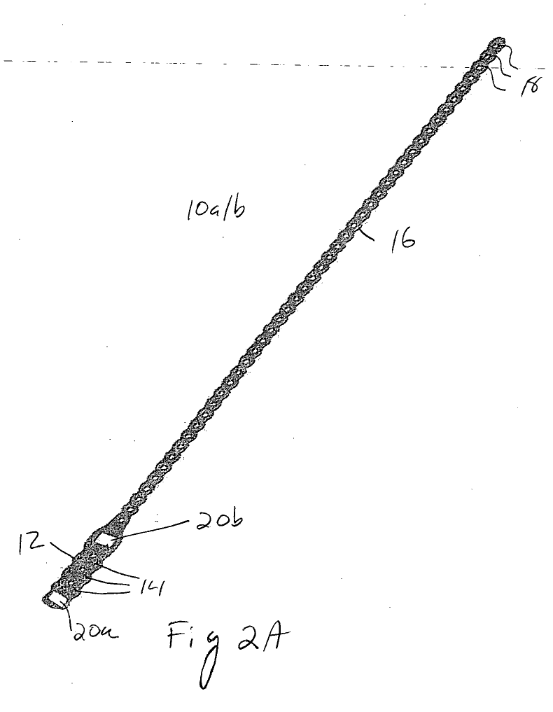

The present invention is a tissue approximation sling device 1 as shown in FIGS. 1A and 1B. Sling device 1 includes a pair of tissue approximation devices 10a/10b slidably interconnected to each other. The tissue approximation devices 10a/b are preferably identical, and are better shown in FIGS. 2A and 2B. Each tissue approximation device 10a/b includes a supportive backing member 12, a plurality of tissue engagement tines 14 extending from the backing member 12, and an elongated extension member or leash 16 extending from the backing member 12. The tines 14 are preferably angled or canted toward the extension member 16 portion of the approximation device. The extension member 16 preferably includes a plurality of through-holes 18 formed therein in a line along the length of the extension member 16. The backing member 12 also includes a pair of apertures 20a/b for engaging with the extension member 16 of the other approximation device.

The sling device 1 of the present invention is assembled by feeding the extension member 16 of approximation device 10b through the apertures 20a/b of approximation device 10a (so that the extension member extends behind much of the backing member 12 of approximation device 10a), and by feeding the extension member 16 of approximation device 10a through aperture 20a of approximation device 10b, as shown in FIGS. 1A and 1B. As the ends of extension members 16 are pulled away from each other, the approximation devices 10a/b slide relative to each other so that backing members 12 slide toward each other. Each of the extension members 16 have scalloped edges to reduce the visibility of the sling device after implementation (long straight lines are easier to see than a scalloped or uneven edges that geometrically break up visibility under the skin).

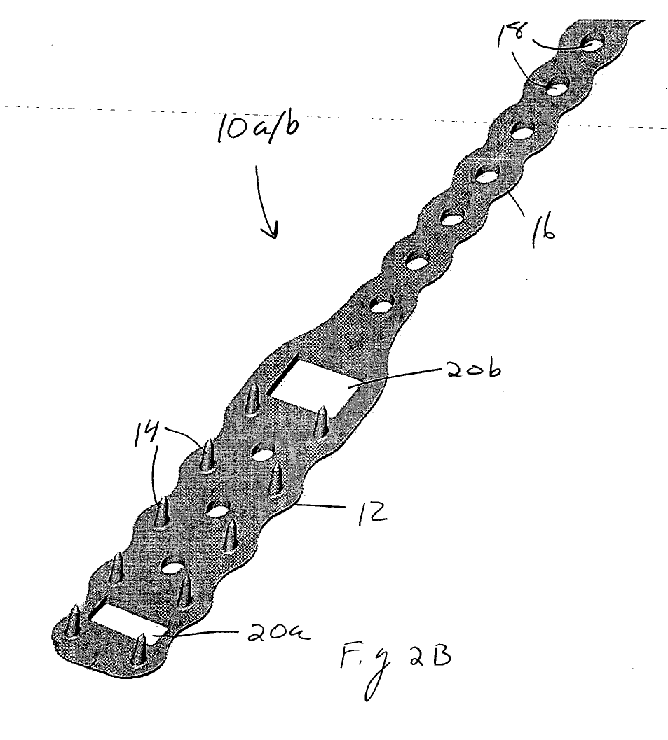

The implementation of the sling device 1 in the repair of the platysma 30 is illustrated in FIG. 3. The sling device is inserted into the neck and over (or under with tines facing up) the platysma muscle 30 so that the tines 14 of the approximation devices 10 engage the desired portions of the platysma 30. The ends of extension members 16 are then pulled up along the mastoid process (to which the sling device will be anchored). By pulling on the extension members 16, the backing members 12 (along with the platysma portions engaged with the tines thereof) are drawn toward each other, until the platysma portions are approximated to their desired locations. At this time, the backing members engaged with the platysma can be secured in their new relative location prior to securing the extension members. The extension members 12 are then anchored to the mastoid process, preferably using screws 32 extending through selected through holes 18. Additionally or alternately, sutures may be used to anchor the extension members to the mastoid fascia, also utilizing the through holes. Excess length of the extension members 16 can then be cut away. By engaging the platysma using a plurality of tines disposed over the areas of the backing members 12, the tension of tissue engagement is broadly dispersed over a wide area. The sliding configuration of the tissue approximation devices 10a/b allows the surgeon to select the degree of tissue approximation after engaging the platysma simply by pulling on the extension members before anchoring. The sling can mobilize, move, or stabilize the selected regions of the platysma, where the approximation forces of the platysma are counteracted by the forces of tissue support to the mastoid fascia or process. The approximation forces of the platysma can also be counteracted through the use of a suture tied so as to fix the two extension members relative to each other. This same function can also be achieved through the use of a separate locking device or through a ratchet mechanism molded into the sling device.

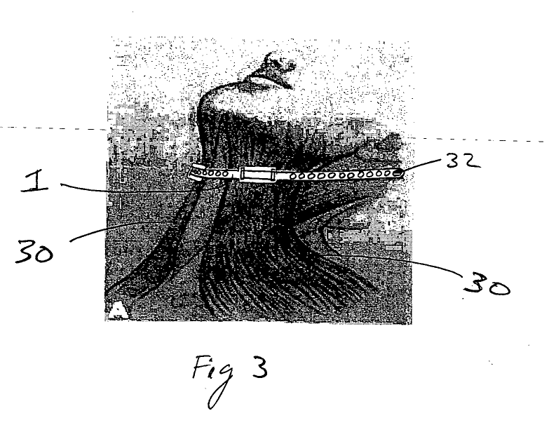

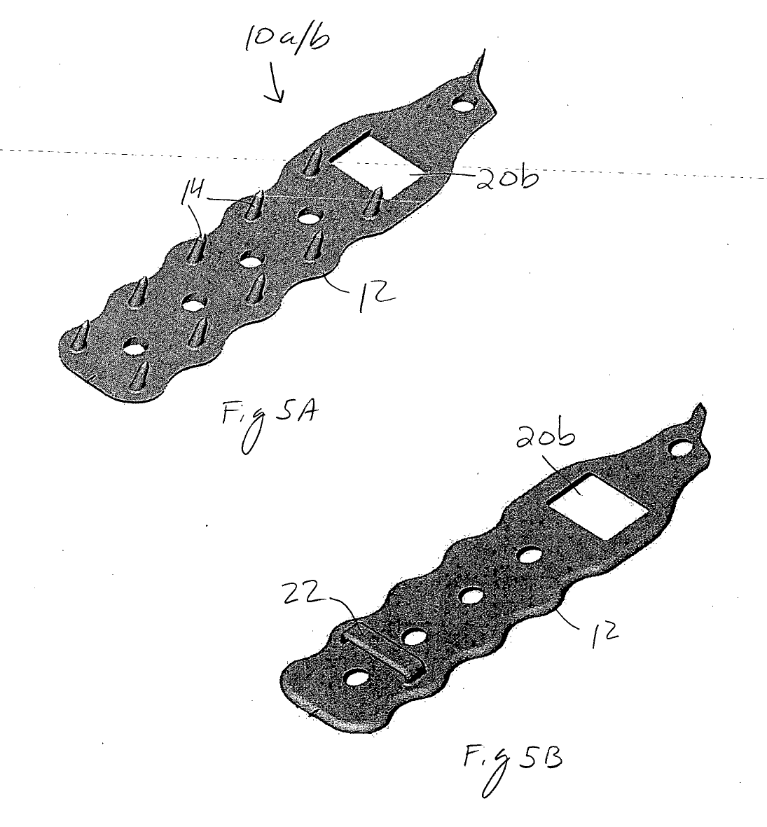

FIGS. 4 and 5A/B illustrate an alternate embodiment of the tissue approximation devices 10a/b, where apertures 20a are replaced by sleeve members 22 formed on the back side of backing members 12. The extension members 16 extend through the sleeve members 22 instead of apertures 20a.

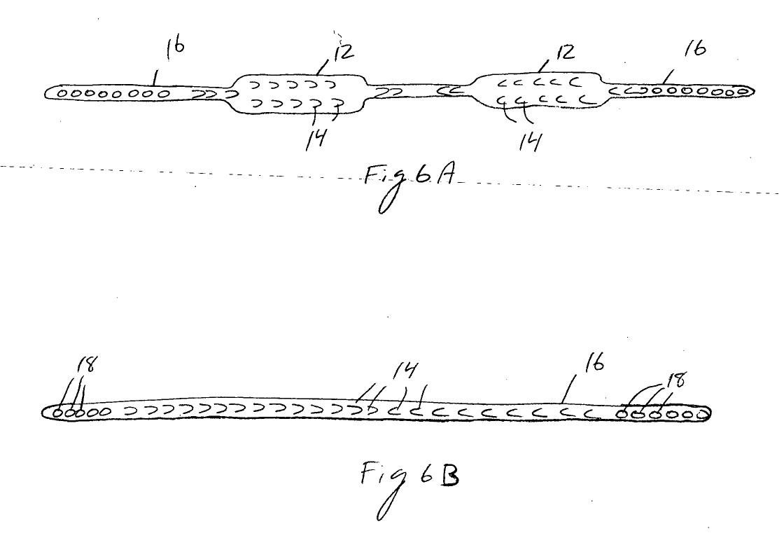

It is to be understood that the present invention is not limited to the embodiment(s) described above and illustrated herein, but encompasses any and all variations falling within the scope of the appended claims. For example, the sling components and/or the mounting screws can be made of biodegradable materials well known in the art or other bio-compatible materials. Additional tines can be formed on the extension members 16, to engage with additional portions of the platysma, and/or to engage with the mastoid process or fascia instead of using screws. The sling of the present invention can be used on other types of tissue, and its application is not limited to the platysma. Lastly, it is within the scope of the present invention to provide a unitary sling device as shown in FIGS. 6A or 6B, where the backing portions 12 are connected together in a non-sliding configuration (FIG. 6A), or tines are simply formed along the length of a single extension member (FIG. 6B).

1. A medical sling device, comprising:
a first tissue approximation device that includes a first supportive backing member, a first plurality of tissue engagement tines extending from the first backing member, and a first elongated extension member extending from the first backing member; and
a second tissue approximation device that includes a second supportive backing member, a second plurality of tissue engagement tines extending from the second backing member, and a second elongated extension member extending from the second backing member;
wherein the first extension member is slidably engaged with the second backing member, and the second extension member is slidably engaged with the first backing member, such that tension pulling on the first and second extension members causes the first and second backing members to slide toward each other.

2. The medical device of claim 1, wherein:
the first plurality of tissue engagement tines are canted toward the first extension member; and
the second plurality of tissue engagement tines are canted toward the second extension member.

3. The medical device of claim 1, wherein the first and second extension members each include a plurality of through-holes formed therein.

4. The medical device of claim 3, wherein for each of the first and second extension members, the plurality of through-holes formed therein are disposed in a line along the length of the extension member.

5. The medical device of claim 1, wherein:
the first backing member includes an aperture through which the second extension member slidably extends; and
the second backing member includes an aperture through which the first extension member slidably extends.

6. The medical device of claim 1, wherein:
the first backing member includes an aperture through which the second extension member slidably extends; and
the second backing member includes a sleeve through which the first extension member slidably extends.

7. The medical device of claim 6, wherein the first backing member further includes a second sleeve through which the second extension member slidably extends.

8. The medical device of claim 1, wherein each of the first and second extension members includes a pair of scalloped edges extending along a length thereof.

9. A method of approximating first and second tissue portions using a medical sling device, wherein the sling device comprises a first tissue approximation device that includes a first supportive backing member, a first plurality of tissue engagement tines extending from the first backing member, and a first elongated extension member extending from the first backing member; and a second tissue approximation device that includes a second supportive backing member, a second plurality of tissue engagement tines extending from the second backing member, and a second elongated extension member extending from the second backing member; wherein the first extension member is slidably engaged with the second backing member, and the second extension member is slidably engaged with the first backing member, the method comprising:
placing the sling device on the first and second tissue portions such that the first plurality of tines engage with a first tissue portion and the second plurality of tines engage with the second tissue portion;
pulling on the first and second extension members such that the first and second backing members slide toward each other drawing the first and second tissue portions toward each other; and
securing the first and second extension members in place.

10. The method of claim 9, wherein the placing of the sling device includes inserting the sling device over a platysma muscle of a patient, and wherein the first and second tissue portions are first and second tissue portions of the platysma muscle.

11. The method of claim 10, wherein the securing of the first and second extension members includes securing the first and second extension members to a mastoid process of the patient.

12. The method of claim 11, wherein the securing of the first and second extension members further includes:
inserting screws through holes formed in the first and second extension members; and
securing the screws to the mastoid process of the patient.

13. The method of claim 11, wherein the securing of the first and second extension members further includes:
inserting sutures through holes formed in the first and second extension members; and
securing the sutures to the mastoid process of the patient.

14. The method of claim 11, further comprising:
cutting off excess portions of the first and second extension members after the securing of the first and second extension members.
